(19) United States
(12) Patent Application Publication
(10) Pub. No.: US 2002/0156277 A1
(43) Pub. Date: Oct. 24, 2002

(54) Synthesis and methods of use of purine analogues and derivatives

(76) Inventors: David B. Fick, Newport Beach, CA (US); Mark M. Foreman, Tustin, CA (US); Alvin J. Glasky, Tustin, CA (US)

Correspondence Address: OPPENHEIMER WOLFF & DONNELLY LLP, 840 NEWPORT CENTER DRIVE, SUITE 700, NEWPORT BEACH, CA, 92660, US

(21) Appl. No.: 09/839,290

(22) Filed: Apr. 20, 2001

(51) Int. Cl. C07D 473/04

(52) U.S. Cl. 544/262; 544/269; 544/271; 544/277; 544/60; 544/238; 544/118

(57) ABSTRACT

A purine derivative or analogue comprises a 9-atom bicyclic moiety, moiety A, linked through a linker L to a moiety B, where B is a carboxylic acid, a carboxylic acid ester, or a moiety of the structure N(Y₁)—D, where Y₁ can be one of a variety of substituents, including hydrogen or alkyl, and D is a moiety that enhances the pharmacological effects, promotes absorption or blood-brain barrier penetration of the derivative or analogue. The moiety A has a six-membered ring fused to a five-membered ring. The moiety A can have one, two, or three nitrogen atoms in the five membered ring and has two nitrogen atoms in the six-membered ring. The moiety A can be a purine moiety. The moiety B can be one of a variety of moieties, including moieties having nootropic activity or other biological or physiological activity.

### BACKGROUND OF THE INVENTION

1. Field of the Invention

The present invention is directed to purine derivatives and analogues, particularly purine derivatives or analogues in which the purine derivative or analogue is covalently linked to another moiety to form a bifunctional conjugate.

2. General Background and State of the Art

Diseases and degenerative conditions of the central nervous system are among the most severe, long-lasting, and chronic diseases and conditions affecting man. Although much research has been done on such diseases and conditions, effective treatment remains elusive. These diseases and conditions include Alzheimer's disease, Huntington's disease, amyotrophic lateral sclerosis (Lou Gehrig's disease), Parkinson's disease, multiple sclerosis, stroke, and other neurodegenerative disorders, which may be genetic, spontaneous or drug-induced.

There is therefore a need for improved compounds and methods for treating such conditions. The need for such improved compounds and methods has been increased by the discovery that such compounds are capable of increasing neuronal function, stimulating nerve growth or regeneration and can act through the induction of neurotrophic factors such as nerve growth factor, NT-3, brain-derived neurotrophic factor (BDNF), or ciliary neurotrophic factor (CNTF). Such compounds may stimulate nerve regeneration or neurogenesis in the peripheral nervous system or central nervous system, or neuroprotection, and may therefore be of use in the treatment of the diseases and conditions referred to above.

There is therefore a particular need for the development of additional compounds that have improved activity in stimulating neuronal function, regeneration, neurogenesis, and that have neuroprotective activity. There is further a need for compounds that have activities that provide treatment for or relief from symptoms of diseases and conditions such as Alzheimer's disease, Huntington's disease, Parkinson's disease, multiple sclerosis, stroke and other neurodegenerative disorders, which may be genetic, spontaneous or drug-induced. Examples of these symptoms include reduced cognition, emotional control, and sensory or motor function. There is a particular need for the development of new compounds that have improved bioavailability. There is a further need for compounds with a greater degree of activity as measured by a dose-response curve assay and for compounds with a different spectrum of activities.

### INVENTION SUMMARY

One aspect of the present invention is purine derivatives and analogues. In general, a purine derivative or analogue of the present invention has the schematic structure:

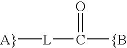

where:

(1) A is a substituted or unsubstituted 9-atom bicyclic moiety in which the five-membered ring has 1 to 3 nitrogen atoms, the bicyclic moiety having the structure of formula (I)

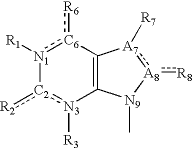

where:

(a) if the bond between N₁ and C₆ is a single bond, then the bond between C₆ and R₆ is a double bond, R₆ is O or S, and R₁ is hydrogen, alkyl, aralkyl, cycloalkyl, or heteroaralkyl;

(b) if the bond between N₁ and C₆ is a double bond, then the bond between C₆ and R₆ is a single bond, R₁ is not present, and R₆ is hydrogen, halo, amino, OQ₁, SQ₁, NHNH₂, NHOQ₁, NQ₁Q₂, or NHQ₁, where Q₁, and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₁ and Q₂ are present together and are alkyl, they can be taken together to form a 5 or 6 member ring which may contain 1 other heteroatom which can be N, O, or S, of which the N may be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S,;

(c) if the bond between C₂ and N₃ is a single bond, then the bond between C₂ and R₂ is a double bond, R₂ is O or S, and R₃ is hydrogen or alkyl;

(d) if the bond between C₂ and N₃ is a double bond, then the bond between C₂ and R₂ is a single bond, R₃ is not present, and R₂ is hydrogen, alkyl, aralkyl, cycloalkyl, heteroaralkyl, halo, amino, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₁ and Q₂ are present together and are alkyl, they can be taken together to form a 5 or 6 member ring which may contain 1 other heteroatom which can be N, O, or S, of which the N may be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S,;

(e) A₇ and A₈ are C or N;

(i) if A₇ and A₈ are both C and the bond between A₇ and A₈ is a single bond, then the bond between A₈ and R₈ is two single bonds to two hydrogen atoms or is a double bond in which R₈ is O or S and R₇ is two hydrogen atoms;

(ii) if A₇ and A₈ are both C and the bond between A₇ and A₈ is a double bond, then R₇ is hydrogen, the bond between A₈ and R₈ is a single bond and R₈ is hydrogen, halo, alkyl, alkenyl, aryl, aralkyl, aralkenyl, heteroaryl, heteroaralkyl, or heteroaralkenyl;

(iii) if A₇ and A₈ are both N, then the bond between A₇ and A₈ is a double bond and R₇ and R₈ are not present;

(iv) if A₇ is C and A₈ is N, then the bond between A₇ and A₈ is a double bond, R₇ is hydrogen, and R₈ is not present;

(v) if A₇ is N, A₈ is C, and the bond between A₇ and A₈ is a double bond, then R₇ is not present, the bond between A₈ and R₈ is a single bond, and R₈ is hydrogen, halo, alkyl, alkenyl, aryl, aralkyl, aralkenyl, heteroaryl, heteroaralkyl, or heteroaralkenyl;

(vi) if A₇ is N, A₈ is C, and the bond between A₇ and A₈ is a single bond, then R₇ is hydrogen, alkyl, aryl, aralkyl, heteroaryl, or heteroaralkyl, the bond between A₈ and R₈ is a double bond, and R₈ is O or S;

(f) N₉ is bonded to L;

(2) L is a hydrocarbyl moiety of 1 to 6 carbon atoms that can be cyclic, with the hydrocarbyl moiety being optionally substituted with one or more substituents selected from the group consisting of lower alkyl, amino, hydroxy, lower alkoxy, lower alkylamino, lower alkylthio and oxo; and

(3) B is —OZ or N(Y₁)—D, where Z is hydrogen, alkyl, aryl, heteroaryl, cycloalkyl, aralkyl, or heteroaralkyl, D is a moiety that promotes absorption of the derivative or analogue, and Y₁ is hydrogen, alkyl, aryl, heteroaryl, aralkyl, or heteroaralkyl, which, when taken with D, can form a cyclic 5- or 6-membered saturated structure which can contain one other heteroatom which can be O, N, or S, of which N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;

with the proviso that A does not have the structure of an unsubstituted guanine or hypoxanthine.

Typically, A is a purine moiety.

B is either: (i) a moiety with the structure —OZ, where Z is hydrogen, alkyl, aryl, heteroaryl, cycloalkyl, aralkyl, or heteroaralkyl; or (ii) a moiety with the structure —N(Y₁)-D, where D is a moiety that promotes absorption of the derivative or analogue that can be substituted as indicated above.

If B is a moiety with the structure —OZ, it is a carboxylic acid or a carboxylic acid ester. Typically, if B is a carboxylic acid ester, the moiety Z is one of methyl, ethyl, propyl, butyl, or isobutyl. More typically, Z is hydrogen or ethyl.

If B is a moiety with the structure —N(Y₁)—D, Y₁ is hydrogen, alkyl, aryl, heteroaryl, aralkyl, or heteroaralkyl, which, when taken with D, can form a cyclic 5- or 6-membered saturated ring which can contain one other heteroatom which can be O, N, or S, of which N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, heteroaralkylaminocarbonyl, in which the alkyl portions can be cyclic and can contain from 1 to 3 heteroatoms which can be N, O, or S. Typically, Y₁ is hydrogen or lower alkyl. Most typically, Y₁ is hydrogen.

Typically, the purine derivative or analogue has a logP of from about 1 to about 4 to enhance bioavailability and central nervous system (CNS) penetration. Using this guideline, one of ordinary skill in the art can choose the appropriate moieties B for a particular moiety A in order to ensure the bioavailability and CNS penetration of a purine analogue or derivative according to the present invention. For example, if a highly hydrophobic moiety A is chosen, with particularly hydrophobic substituents on the purine moiety, then a more hydrophilic moiety B can be used.

In one alternative, B is a moiety containing at least one carboxyl, carboxamide, carboxyl ester, or carbonyl function.

In another alternative, B is a cyclic or acyclic moiety containing at least one hydroxyl, primary amino, secondary amino, tertiary amino, sulfhydryl, or sulfonamidyl function.

Particular examples of purine derivatives and analogues according to the present invention include: (1) a purine derivative or analogue that is 4-[3-(1-benzyl-6-oxo-1,6-dihydropurin-9-yl)propionylamino]benzoic acid ethyl ester; (2) a purine derivative or analogue that is 4-[3-(1-butyl-6-oxo-1,6-dihydropurin-9-yl)propionylamino]benzoic acid ethyl ester; (3) a purine derivative or analogue that is 4-[3-(1-methyl-6-oxo-1,6-dihydropurin-9-yl)propionylamino]benzoic acid ethyl ester; (4) a purine derivative or analogue that is 4-[3-(1-(2-dimethylaminoethyl)-6-oxo-1,6-dihydropurin-9-yl)propionylamino]benzoic acid ethyl ester; (5) a purine derivative or analogue that is 4-[3-(2,6-dioxo-1,2,3,6-tetrahydropurin-9-yl)propionylamino]benzoic acid ethyl ester; (6) a purine derivative or analogue that is 4-[3-(6-methoxypurin-9-yl)propionylamino]benzoic acid ethyl ester; (7) a purine derivative or analogue that is 4-[3-(6-dimethylaminopurin-9-yl)propionylamino]benzoic acid ethyl ester; (8) a purine derivative or analogue that is 4-[3-(2-amino-6-chloropurin-9-yl)propionylamino]benzoic acid ethyl ester; (9) a purine derivative or analogue that is 4-[2-(6-oxo-2-thioxo-1,2,3,6-tetrahydropurin-9-yl)propionylamino]benzoic acid ethyl ester; (10) a purine derivative or analogue that is 4-[2-(2-butyl-6-oxo-1,6-dihydropurin-9-yl)propionylamino]benzoic acid ethyl ester; (11) a purine derivative or analogue that is 4-[2-(6-oxo-2-phenyl-1,6-dihydropurin-9-yl)propionylamino]benzoic acid ethyl ester; (12) a purine derivative or analogue that is 4-{[3-(6-chloropurin-9-yl)propionyl]methylamino}benzoic acid methyl ester; (13) a purine derivative or analogue that is 3-(1-benzyl-6-oxo-1,6-dihydropurin-9-yl)-N-[3-(2-oxopyrrolidin-1-yl)propyl]propionamide; (14) a purine derivative or analogue that is 3-(1-benzyl-6-oxo-1,6-dihydropurin-9-yl)-N-{2-[2-(2-oxopyrrolidin-1-yl)acetylamino]ethyl}propionamide; (15) a purine derivative or analogue that is N-3-(2-oxopyrrolidin-1-yl)propyl]-3-(6-oxo-2-thioxo-1,2,3,6-tetrahydropurin-9-yl) propionamide; and (16) a purine derivative or analogue that is 3-(1-benzyl-6-oxo-1,6-dihydropurin-9-yl)-N-(3-morpholin-4-yl-propyl) propionamide.

Another aspect of the present invention is methods of use of the purine derivatives and analogues described above. One aspect of a method of use of purine derivatives and analogues according to the present invention is a method of stimulating neuronal function such as improved cognition, involving neuronal regeneration or axo-dendritic complexity in the central and peripheral nervous systems comprising the step of administering an effective amount of a purine derivative or analogue according to the present invention to the mammal. Another aspect of a method of use of purine derivatives and analogues according to the present invention is a method of stimulating neuronal function such as improved cognition, involving by initiating neurogenesis in the central nervous system of a mammal comprising the step of administering an effective amount of a purine derivative or analogue according to the present invention to the mammal. Yet another aspect of a method of use of purine derivatives and analogues according to the present invention is a method of stimulating neuronal function involving mechanism associated with neuroprotection in the central or peripheral nervous system of a mammal comprising the step of administering an effective amount of a purine derivative or analogue according to the present invention to the mammal.

Other methods according to the present invention include a method of stimulating neuronal function involving either inhibition of the formation of the amyloid beta-peptide (Aβ) or stimulating the formation of the secreted derivative of the amyloid precursor protein known as sAPPα by administering to a patient with a neurological disease or a patient at risk of developing a neurological disease an effective quantity of a purine derivative or analogue according to the present invention.

Another aspect of the present invention is pharmaceutical compositions. A pharmaceutical composition according to the present invention comprises: (1) an effective amount of a purine derivative or analogue according to the present invention; and (2) a pharmaceutically acceptable carrier.

### BRIEF DESCRIPTION OF THE DRAWINGS

The following invention will become better understood with reference to the specification, appended claims, and accompanying drawings where:

FIG. 1 is a table (Table 1) depicting a number of the purine derivatives or analogues according to the present invention together with the minimum effective dose in the passive avoidance test for nootropic activity in mice with either intraperitoneal or oral administration.

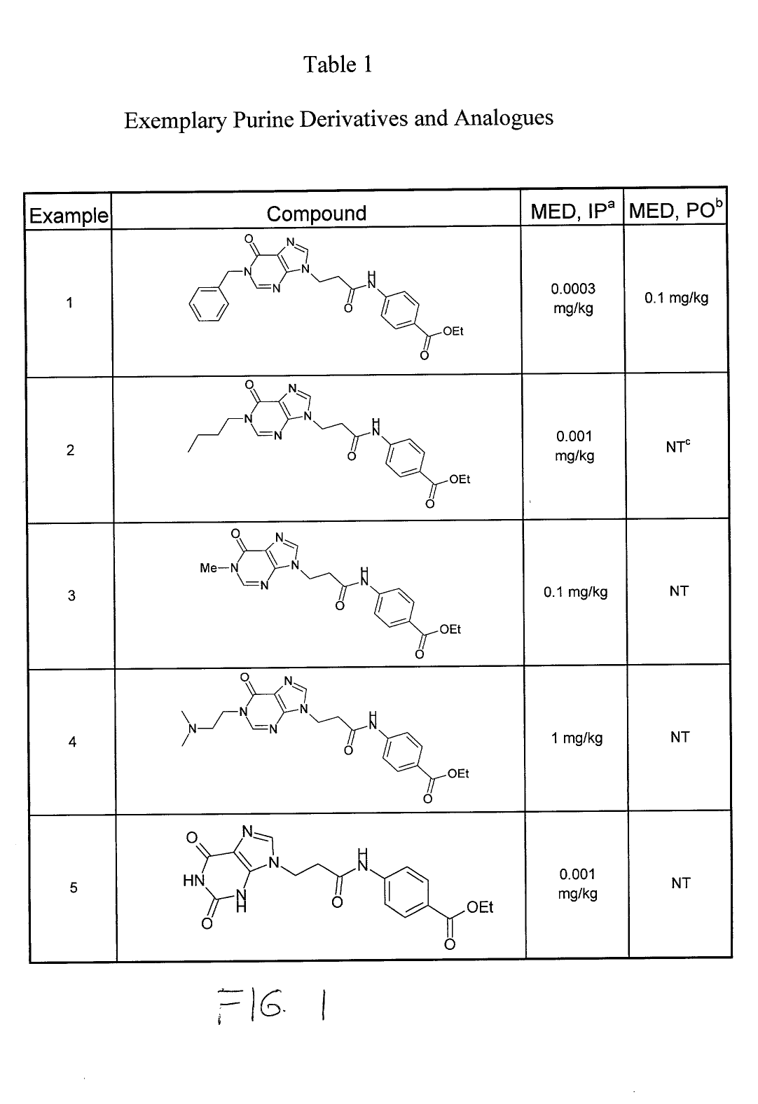

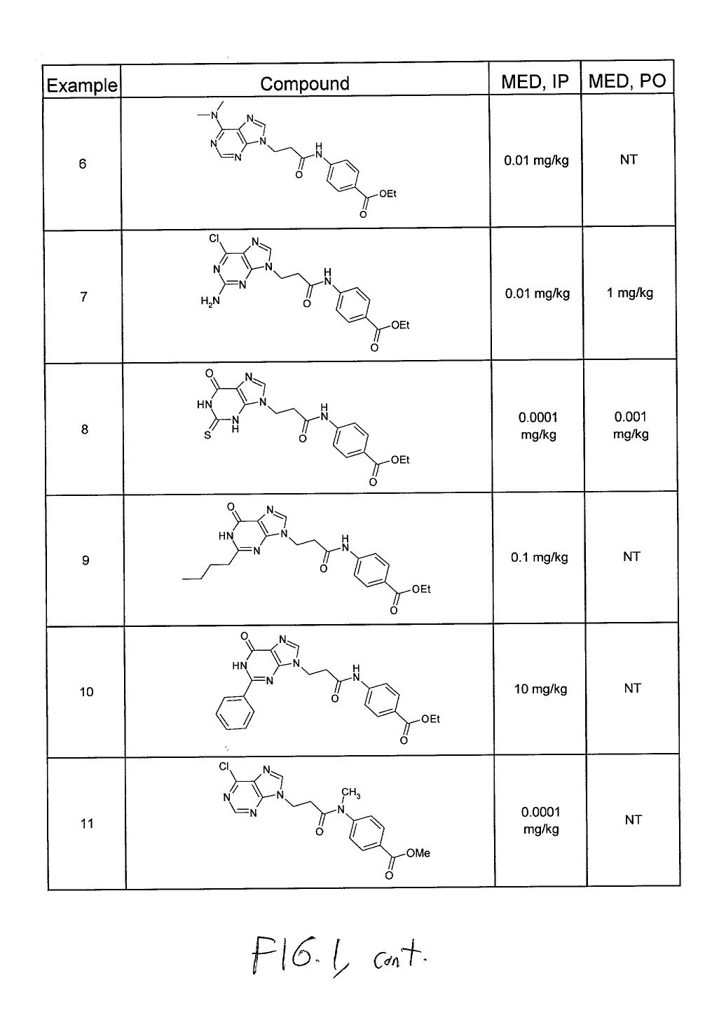

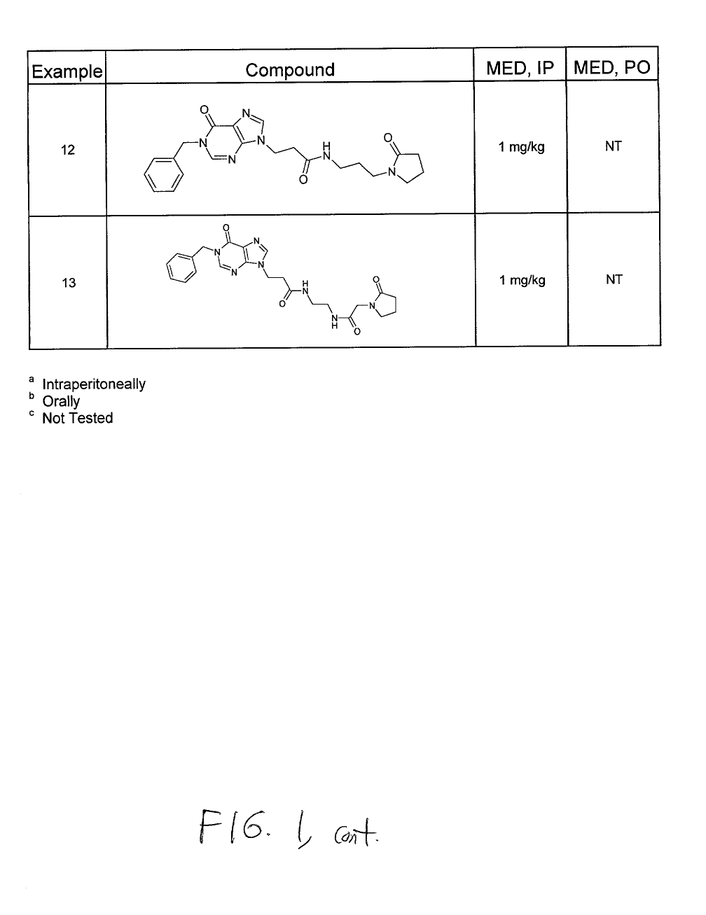

### DETAILED DESCRIPTION OF THE PREFERRED EMBODIMENTS

I. Purine Derivatives and Analogues

One aspect of the present invention is purine derivatives and analogues.

In its most general aspect, a purine derivative or analogue according to the present invention has the schematic structure:

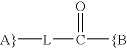

where:

(1) A is a substituted or unsubstituted 9-atom bicyclic moiety in which the five-membered ring has 1 to 3 nitrogen atoms, the bicyclic moiety having the structure of formula (I)

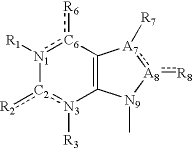

where:

(a) if the bond between N₁ and C₆ is a single bond, then the bond between C₆ and R₆ is a double bond, R₆ is O or S, and R₁ is hydrogen, alkyl, aralkyl, cycloalkyl, or heteroaralkyl;

(b) if the bond between N₁ and C₆ is a double bond, then the bond between C₆ and R₆ is a single bond, R₁ is not present, and R₆ is hydrogen, halo, amino, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₁ and Q₂ are present together and are alkyl, they can be taken together to form a 5 or 6 member ring which may contain 1 other heteroatom which can be N, O, or S, of which the N may be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S,;

(c) if the bond between C₂ and N₃ is a single bond, then the bond between C₂ and R₂ is a double bond, R₂ is O or S, and R₃ is hydrogen or alkyl;

(d) if the bond between C₂ and N₃ is a double bond, then the bond between C₂ and R₂ is a single bond, R₃ is not present, and R₂ is hydrogen, alkyl, aralkyl, cycloalkyl, heteroaralkyl, halo, amino, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₁ and Q₂ are present together and are alkyl, they can be taken together to form a 5 or 6 member ring which may contain 1 other heteroatom which can be N, O, or S, of which the N may be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S,;

(e) A₇ and A₈ are C or N;

(i) if A₇ and A₈ are both C and the bond between A₇ and A₈ is a single bond, then the bond between A₈ and R₈ is two single bonds to two hydrogen atoms or is a double bond in which R₈ is O or S and R₇ is two hydrogen atoms;

(ii) if A₇ and A₈ are both C and the bond between A₇ and A₈ is a double bond, then R₇ is hydrogen, the bond between A₈ and R₈ is a single bond and R₈ is hydrogen, halo, alkyl, alkenyl, aryl, aralkyl, aralkenyl, heteroaryl, heteroaralkyl, or heteroaralkenyl;

(iii) if A₇ and A₈ are both N, then the bond between A₇ and A₈ is a double bond and R₇ and R₈ are not present;

(iv) if A₇ is C and A₈ is N, then the bond between A₇ and A₈ is a double bond, R₇ is hydrogen, and R₈ is not present;

(v) if A₇ is N, AB is C, and the bond between A₇ and A₈ is a double bond, then R₇ is not present, the bond between A₈ and R₈ is a single bond, and R₈ is hydrogen, halo, alkyl, alkenyl, aryl, aralkyl, aralkenyl, heteroaryl, heteroaralkyl, or heteroaralkenyl;

(vi) if A₇ is N, A₈ is C, and the bond between A₇ and A₈ is a single bond, then R₇ is hydrogen, alkyl, aryl, aralkyl, heteroaryl, or heteroaralkyl, the bond between A₈ and R₈ is a double bond, and R₈ is O or S;

(f) N₉ is bonded to L;

(2) L is a hydrocarbyl moiety of 1 to 6 carbon atoms that can be cyclic, with the hydrocarbyl moiety being optionally substituted with one or more substituents selected from the group consisting of lower alkyl, amino, hydroxy, lower alkoxy, lower alkylamino, lower alkylthio, and oxo; and

(3) B is —OZ or N(Y₁)—D, where Z is hydrogen, alkyl, aryl, heteroaryl, cycloalkyl, aralkyl, or heteroaralkyl, D is a moiety that promotes absorption of the derivative or analogue, and Y₁ is hydrogen, alkyl, aryl, heteroaryl, aralkyl, or heteroaralkyl, which, when taken with D, can form a cyclic 5- or 6-membered saturated structure which can contain one other heteroatom which can be O, N, or S, of which N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;

with the proviso that A does not have the structure of an unsubstituted guanine or hypoxanthine.

Typically, Y₁ is hydrogen or lower alkyl. Most typically, Y₁ is hydrogen.

Typically, the purine derivative or analogue has a logP of from about 1 to about 4 to enhance bioavailability and central nervous system (CNS) penetration. Using this guideline, one of ordinary skill in the art can choose the appropriate moieties B for a particular moiety A in order to ensure the bioavailability and CNS penetration of a purine analogue or derivative according to the present invention. For example, if a highly hydrophobic moiety A is chosen, with particularly hydrophobic substituents on the purine moiety, then a more hydrophilic moiety B can be used.

In many examples, in a purine analogue or derivative according to the present invention, the moiety B has a biological, physiological, or pharmacological function, and the purine analogue or derivative is referred to as a “bifunctional conjugate.” However, it is not required in purine analogues or derivatives according to the present invention that the moiety B have a biological, physiological, or pharmacological function. The moiety B can serve as a carrier to improve bioavailability or to optimize the physical characteristics of the molecule without having a separate biological, physiological function, or pharmacological function.

In many purine analogues or derivatives according to the present invention, the moiety B includes a p-aminobenzoic acid, a p-aminobenzoic acid ester, a m-aminobenzoic acid, or a m-aminobenzoic acid ester. However, the moiety B can include other groups.

Typically A is a purine moiety in which A₇ is carbon and A₈ is nitrogen. The purine moiety can be variously substituted so that it has the structure of a naturally-occurring purine such as xanthine, adenine, or another naturally-occurring or synthetic purine other than guanine or hypoxanthine. When A is a purine moiety, the result is a purine derivative; when A is other than a purine moiety, then the result is a purine analogue.

When A is a purine moiety, typical unsubstituted or substituted purine moieties include, but are not limited to, 1-methylhypoxanthine, 6-methoxypurine, N,N-dimethyladenine, 1-dimethylaminoethylhypoxanthine, 1-butylhypoxanthine, 1-benzylhypoxanthine, thioxanthine, xanthine, 2-amino-6-chloropurine, 6-chloropurine, 2-butylhypoxanthine, 1-cyclopropylmethylhypoxanthine, or 2-phenylhypoxanthine. Other unsubstituted or substituted purine moieties can be used.

The linker L is described further below. L is a hydrocarbyl moiety of 1 to 6 carbon atoms that can be cyclic, with the hydrocarbyl moiety being optionally substituted with one or more substituents selected from the group consisting of lower alkyl, amino, hydroxy, lower alkoxy, lower alkylamino, lower alkythio, and oxo.

In accordance with the present invention, and as used herein, the following terms, when appearing alone or as part of a moiety including other atoms or groups, are defined with the following meanings, unless explicitly stated otherwise. In addition, all groups described herein can be optionally substituted unless such substitution is excluded. The term “alkyl,” as used herein at all occurrences, refers to saturated aliphatic groups including straight-chain, branched-chain, and cyclic groups, all of which can be optionally substituted. Preferred alkyl groups contain 1 to 10 carbon atoms. Suitable alkyl groups include methyl, ethyl, and the like, and can be optionally substituted. The term “alkenyl,” as used herein at all occurrences, refers to unsaturated groups which contain at least one carbon-carbon double bond and includes straight-chain, branched-chain, and cyclic groups, all of which can be optionally substituted. Preferable alkenyl groups have 2 to 10 carbon atoms. The term “alkoxy” refers to the ether —O-alkyl, where alkyl is defined as as above. The term “aryl” refers to aromatic groups which have at least one ring having a conjugated π-electron system and includes carbocyclic aryl and biaryl, both of which may be optionally substituted. Preferred aryl groups have 6 to 10 carbon atoms. The term “aralkyl” refers to an alkyl group substituted with an aryl group. Suitable aralkyl groups include benzyl and the like; these groups can be optionally substituted. The term “aralkenyl” refers to an alkenyl group substituted with an aryl group. The term “heteroaryl” refers to carbon-containing 5-14 membered cyclic unsaturated radicals containing one, two, three, or four O, N, or S heteroatoms and having 6, 10, or 14 π-electrons delocalized in one or more rings, e.g., pyridine, oxazole, indole, thiazole, isoxazole, pyrazole, pyrrole, each of which can be optionally substituted as discussed above. The term “sulfonyl” refers to the group —S(O₂)—. The term “alkanoyl” refers to the group —C(O)Rg, where Rg is alkyl. The term “aroyl” refers to the group —C(O)Rg, where Rg is aryl. Similar compound radicals involving a carbonyl group and other groups are defined by analogy. The term “aminocarbonyl” refers to the group —NHC(O)—. The term “oxycarbonyl” refers to the group —OC(O)—. The term “heteroaralkyl” refers to an alkyl group substituted with a heteroaryl group. Similarly, the term “heteroaralkenyl” refers to an alkenyl group substituted with a heteroaryl group. As used herein, the term “lower,” in reference to an alkyl or the alkyl portion of an another group including alkyl, is defined as a group containing one to six carbon atoms. The term “optionally substituted” refers to one or more substituents that can be lower alkyl, aryl, amino, hydroxy, lower alkoxy, aryloxy, lower alkylamino, arylamino, lower alkylthio, arylthio, or oxo, in some cases, other groups can be included, such as cyano, acetoxy, or halo. The term “halo” refers generally to fluoro, chloro, bromo, or iodo; more typically, “halo” refers to chloro.

A preferred linker has the structure —(CH₂)ₙ— wherein n is an integer from 1 to 6. As detailed below, for most preferred embodiments of purine derivatives or analogues according to the present invention, a preferred linker has n equal to 2 or 3. Particular examples of purine derivatives or analogues according to the present invention follow.

A number of purine derivatives or analogues according to the present invention are optically active, owing to the presence of chiral carbons or other centers of asymmetry. In cases where purine derivatives or analogues according to the present invention are optically active, all of the possible enantiomers or diastereoisomers are included unless otherwise indicated despite possible differences in activity.

Particularly preferred purine moieties for the moiety A are described below.

One example of a purine moiety for the moiety A is a purine moiety of Formula (II), below, in which:

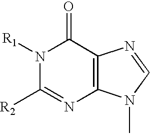

R₁ is selected from the group consisting of hydrogen, alkyl, aralkyl, cycloalkyl, heteroaralkyl, and

R₂ is selected from the group consisting of hydrogen, alkyl, aralkyl, cycloalkyl, heteroaralkyl, halo, amino, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₁ and Q₂ are present together and are alkyl, they can be taken together to form a 5 or 6 member ring which may contain 1 other heteroatom which can be N, O, or S, of which the N may be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S, with the proviso that both R₁ and R₂ are not hydrogen and that R₁ is not hydrogen when R₂ is amino.

The purine moiety of formula (II) is a hypoxanthine or a guanine derivative but excludes unsubstituted hypoxanthine, in which R₁ and R₂ are hydrogen, and unsubstituted guanine, in which R₁ is hydrogen and R₂ is amino.

In one particularly preferred embodiment, R₁ is butyl and R₂ is hydrogen.

In another preferred embodiment, R₁ is benzyl and R₂ is hydrogen.

In another preferred embodiment, R₁ is dimethylaminoethyl and R₂ is hydrogen.

In another preferred embodiment, R₁ is cyclopentyl and R₂ is hydrogen.

In another preferred embodiment, R₁ is cyclohexylmethyl and R₂ is hydrogen.

In another preferred embodiment, R₁ is cyclopropylmethyl and R₂ is hydrogen.

In another preferred embodiment, R₁ is hydrogen and R₂ is phenyl.

In another preferred embodiment, R₁ is hydrogen and R₂ is trifluoromethyl

In another preferred embodiment, R₁ is hydrogen and R₂ is butyl.

In another preferred embodiment, R₁ is butyl and R₂ is butyl.

In another preferred embodiment, R₁ is hydrogen and R₂ is methyl.

In another preferred embodiment, R₁ is hydrogen and R₂ is phenylamino.

Another example of a purine moiety according to the present invention is the purine moiety of Formula (III), below, in which:

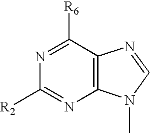

R₂ is selected from the group consisting of hydrogen, halo, amino, OQ₃, SQ₃, NHNH₂, NHOQ₃, NQ₃Q₄, or NHQ₃, where Q₃ and Q₄ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, and heteroaralkylsulfonyl in which the alkyl portions can be cyclic and can contain from 1 to 3 heteroatoms which can be N, O, or S, and when Q₃ and Q₄ are present together and are alkyl, they can be taken together to form a 5- or 6-membered ring which can contain one other heteroatom which can be N, O, or S, of which the N can be further substituted with Y₃, where Y₃ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S; and

R₆ is selected from the group consisting of hydrogen, halo, amino, OQ₅, SQ₅, NHNH₂, NHOQ₅, NQ₅Q₆, or NHQ₅, where Q₅ and Q₆ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, and heteroaralkylsulfonyl in which the alkyl portions can be cyclic and can contain from 1 to 3 heteroatoms which can be N, O, or S, and when Q₅ and Q₆ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S.

In one preferred example of this embodiment, R₂ is hydrogen and R₆ is —NH₂ or —N(CH₃)₂.

In another preferred example of this embodiment, R₂ is hydrogen and R₆ is Cl.

In yet another preferred example of this embodiment, R₂ is —NH₂ and R₆ is Cl.

Another example of a purine moiety according to the present invention is the purine moiety of Formula (IV), below, in which:

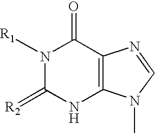

R₁ is hydrogen, alkyl, aralkyl, cycloalkyl, or heteroaralkyl.

R₂ is O or S.

Preferably, R₁ is hydrogen.

The purine moiety of Formula (IV) is a xanthine or thioxanthine moiety.

In addition to these examples of moieties suitable as moiety A, other moieties can serve as moiety A, including moieties with two or three nitrogen atoms or moieties with substituents at R₈.

In general, purine derivatives and analogues that are within the scope of the present invention also include salts and prodrug esters of these purine derivatives and analogues. It is well known that organic compounds, including purines and other components of these purine derivatives and analogues have multiple groups that can accept or donate protons, depending upon the pH of the solution in which they are present. These groups include carboxyl groups, hydroxyl groups, amino groups, sulfonic acid groups, and other groups known to be involved in acid-base reactions. The recitation of a purine derivative or analogue according to the present invention includes such salt forms as occur at physiological pH or at the pH of a pharmaceutical composition unless specifically excluded.

Similarly, prodrug esters can be formed by reaction of either a carboxyl or a hydroxyl group on the purine derivative or analogue with either an acid or an alcohol to form an ester. Typically, the acid or alcohol includes a lower alkyl group such as methyl, ethyl, propyl, isopropyl, butyl, isobutyl, and tertiary butyl. These groups can be substituted with substituents such as hydroxy, halo, or other substituents. Such prodrugs are well known in the art and need not be described further here. The prodrug is converted into the active compound by hydrolysis of the ester linkage, typically by intracellular enzymes. Other suitable groups that can be used to form prodrug esters are well known in the art.

As indicated above, the linker L is a hydrocarbyl moiety of 1 to 6 carbon atoms that can be cyclic, with the hydrocarbyl moiety being optionally substituted with one or more substituents selected from the group consisting of lower alkyl, amino, hydroxy, lower alkoxy, lower alkylamino, lower alkylthio, and oxo. Preferably, the linker L has the structure —(CH₂)ₙ— wherein n is an integer from 1 to 6. As detailed below, for most preferred embodiments of bifunctional conjugates according to the present invention, a preferred linker has n equal to 2 or 3.

The moiety B is either: (i) —OZ, where Z is hydrogen, alkyl, aryl, heteroaryl, cycloalkyl, aralkyl, or heteroaralkyl; or (ii) N(Y₁)—D, where D is a moiety that promotes absorption of the derivative or analogue, and Y₁ is hydrogen, alkyl, aryl, heteroaryl, aralkyl, heteroaralkyl, which, when taken with D, can form a cyclic 5- or 6-membered saturated ring which can contain one other heteroatom which can be O, N, or S, of which N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, heteroaralkylaminocarbonyl, in which the alkyl portions can be cyclic and can contain from 1 to 3 heteroatoms which can be N, O, or S. Typically, Y₁ is hydrogen. Where the moiety B is —OZ, the moiety B is a carboxylic acid or carboxylic acid or ester. Typically, where B is a carboxylic acid ester, the moiety Z is a lower alkyl, such as methyl, ethyl, butyl, propyl, or isopropyl.

In one alternative, the moiety D, as described above, is a moiety having at least one polar, charged, or hydrogen-bond-forming group to improve the metabolic and bioavailability properties of the purine derivative or analogue. The moiety D can be, but is not limited to, a moiety with physiological or biological activity such as nootropic activity. In one alternative, the moiety D can be a moiety containing at least one carboxyl, carboxamide, carboxyl ester, or carbonyl function. In another alternative, the moiety D can be a moiety containing at least one hydroxyl, primary amino, secondary amino, tertiary amino, sulfhydryl, or sulfonamidyl function. The moiety D can be cyclic or acyclic. Preferred examples of the moiety D are described below.

When the moiety D is a cyclic or acyclic moiety containing at least one carbonyl, carboxamide, carboxyl ester, or carbonyl function, in one preferred example, D is a carboxylic acid or carboxylic acid ester with the structure

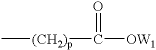

wherein p is an integer from 1 to 6 and W₁ is selected from the group consisting of hydrogen and lower alkyl. Typically, if W₁ is lower alkyl, it is methyl, ethyl, propyl, butyl, or isobutyl. Typically, p is 3. Typically, W₁ is hydrogen or ethyl.

In another preferred example, D and Y₁ are taken together to form a piperazine derivative as described in D. Manetti et al., “Molecular Simplification of 1,4-Diazabicyclo[4.3.0]nonan-9-ones Gives Piperazine Derivatives That Maintain High Nootropic Activity,” J. Med. Chem. 43: 4499-4507 (“Manetti et al. (2000) (II)”). B is an analogue of structure

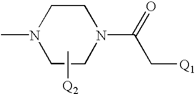

wherein Q₁ is hydrogen, methyl, ethyl, butyl, or propyl, Q₂ is hydrogen or methyl, where, if Q₂ is methyl, it can be located at either of the two possible positions in the piperazine ring.

In another preferred example, D has the structure

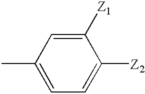

where one of Z₁ and Z₂ is hydrogen, and the other of Z₁ and Z₂ is —COOH or —COOW₁, wherein W₁ is alkyl. Typically, W₁ is selected from the group consisting of methyl, ethyl, propyl, butyl, and isobutyl. Either of Z₁ or Z₂ can be hydrogen. When Z₁ is hydrogen and Z₂ is —COOH, the moiety B is p-aminobenzoic acid (PABA). When Z₁ is —COOH and Z₂ is hydrogen, the moiety B is m-aminobenzoic acid (MABA). When Z₁ is hydrogen and Z₂ is —COOW₁, the moiety B is an ester of p-aminobenzoic acid (PABA). When Z₁ is —COOW₁ and Z₂ is hydrogen, the moiety B is an ester of m-aminobenzoic acid (MABA). Typically, these esters are ethyl esters.

When the moiety D is a moiety that contains at least one hydroxyl, primary amino, secondary amino, tertiary amino, sulfhydryl, or sufonamidyl function, in one preferred example, D is a phenylsulfonamidyl moiety of structure

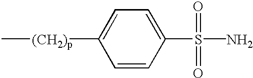

wherein p is an integer from 0 to 6. Typically, p is 2.

In another preferred example, D is an alkylpyridyl moiety of structure

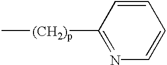

wherein p is an integer from 1 to 6. Typically, p is 1.

In another preferred example, D is a dialkylaminoalkyl moiety of the structure

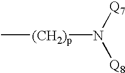

wherein p is an integer from 1 to 6 and Q₇ and Q₈ are alkyl, aralkyl, heteroaralkyl, aryl, heteroaryl, alkanoyl, aroyl, aralkanoyl, heteroaralkanoyl, or heteroaroyl in which the alkyl portions can be cyclic and can contain from 1 to 3 heteroatoms which can be N, O, or S, and when Q₁ and Q₂ are present together and are alkyl, they can be taken together to form a 5 or 6 member ring which may contain 1 other heteroatom which can be N, O, or S, of which the N may be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S.

Where Q₇ and Q₈ can be taken together to form a five or six member ring, the ring is typically pyrrolidine, piperidine, or morpholine. The pyrrolidine ring can be optionally substituted with oxo. The piperidine ring can be optionally substituted with methyl or ethyl. Typically, p is 2 or 3.

In another preferred example, D is an alkylpyrrolidine moiety of the structure

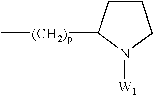

wherein p is an integer from 1 to 6 and W₁ is selected from the group consisting of methyl, ethyl, and propyl. Typically, W₁ is methyl. Typically, p is 2.

Preferably, a purine analogue or derivative according to the present invention has a logP of from about 1 to about 4 in order to optimize bioavailability and CNS penetration of the purine analogue or derivative.

In general, subject to the restrictions indicated above when A is an unsubstituted guanine or hypoxanthine moiety, any moiety A can be combined with any linker L and any moiety B, including the appropriate moiety D, to produce a purine analogue or derivative according to the present invention. However, there exist a number of particularly preferred purine analogues or derivatives according to the present invention. These include the following:

(1) 4-[3-(1-benzyl-6-oxo-1,6-dihydropurin-9-yl)propionylamino]benzoic acid ethyl ester (Example 1);

(2) 4-[3-(1-butyl-6-oxo-1,6-dihydropurin-9-yl)propionylamino]benzoic acid ethyl ester (Example 2);

(3) 4-[3-(1-methyl-6-oxo-1,6-dihydropurin-9-yl)propionylamino]benzoic acid ethyl ester (Example 3);

(4) 4-[3-(1-(2-dimethylaminoethyl)-6-oxo-1,6-dihydropurin-9-yl)propionylamino]benzoic acid ethyl ester (Example 4);

(5) 4-[3-(2,6-dioxo-1,2,3,6-tetrahydropurin-9-yl)propionylamino]benzoic acid ethyl ester (Example 5);

(6) 4-[3-(6-methoxypurin-9-yl)propionylamino]benzoic acid ethyl ester (Example 6);

(7) 4-[3-(6-dimethylaminopurin-9-yl)propionylamino]benzoic acid ethyl ester (Example 7);

(8) 4-[3-(2-amino-6-chloropurin-9-yl)propionylamino]benzoic acid ethyl ester (Example 8);

(9) 4-[2-(6-oxo-2-thioxo-1,2,3,6-tetrahydropurin-9-yl)propionylamino]benzoic acid ethyl ester (Example 9);

(10) 4-[2-(2-butyl-6-oxo-1,6-dihydropurin-9-yl)propionylamino]benzoic acid ethyl ester (Example 10);

(11) 4-[2-(6-oxo-2-phenyl-1,6-dihydropurin-9-yl)propionylamino]benzoic acid ethyl ester (Example 11);

(12) 4-{[3-(6-chloropurin-9-yl)propionyl]methylamino}benzoic acid methyl ester (Example 12);

(13) 3-(1-benzyl-6-oxo-1,6-dihydropurin-9-yl)-N-[3-(2-oxopyrrolidin-1-yl)propyl]propionamide (Example 13);

(14) 3-(1-benzyl-6-oxo-1,6-dihydropurin-9-yl)-N-{2-[2-(2-oxopyrrolidin-1-yl)acetylamino]ethyl}propionamide (Example 14)

(15) N-3-(2-oxopyrrolidin-1-yl)propyl]-3-(6-oxo-2-thioxo-1,2,3,6-tetrahydropurin-9-yl) propionamide (Example 15); and

(16) 3-(1-benzyl-6-oxo-1,6-dihydropurin-9-yl)-N-(3-morpholin-4-yl-propyl) propionamide (Example 16).

II. Methods of Synthesis of Purine Derivatives and Analogues According to the Present Invention

Methods for synthesis of purine derivatives and analogues according to the present invention are modified from those described, for example, in U.S. Pat. No. 5,091,432 to Glasky, incorporated herein by this reference. In one general route in which the purine derivatives and analogues according to the present invention contain a purine moiety, the purine moiety is substituted with a linker which in turn is linked to the moiety B that completes the molecule as described above. This route comprises the steps of: (1) synthesizing an appropriately substituted purine moiety linked to an aliphatic linker in which the linker is terminated with a carboxyl group protected such as with an alkyl ester; (2) hydrolyzing the alkyl ester (or other analogous protecting group) to yield a carboxylic acid; (3) activating the free carboxylic acid by converting it to a nitrophenyl ester; (4) reacting the nitrophenyl ester with an appropriate group that can form an amide or other stable covalent linkage with the carboxyl moiety, with appropriate protection for the moiety reacting with the ester if required; and (5) hydrolyzing the protective group protecting the moiety reacting with the ester to produce the final product.

The length of the aliphatic linker covalently bound to the purine moiety can be varied to vary the distance between the purine moiety and the moiety B in the purine derivative or analogue.

Alternatively, the purine ring can be formed in stages, with the attachment of the linker and the moiety B occuring before the closure of the purine ring. This route involves: (1) the formation of aminocyanacetamide; (2) the reaction of aminocyanacetamide with triethyl orthoformate and acetonitrile to form an amidoester derivative of aminocyanacetamide; (3) the formation of a compound having a reactive amino group on a hydrocarbyl moiety, the hydrocarbyl moiety being linked through an amide group to a derivative of the moiety B, the derivative being protected, such as with an ester group; (4) the reaction of the amidoester with the compound having the reactive amino group on the hydrocarbyl moiety; (5) formation of the six-membered heterocyclic ring of the purine moiety; and (6) hydrolysis of the protecting group, if present, to form the final product.

In one alternative, the step of the formation of the six-membered heterocyclic ring of the purine moiety can be performed by methods analogous to the ring closure of Yamazaki (A. Yamazaki et al., “Synthesis of Guanosine and Its Derivatives from 5-Amino-1-β-D-Ribofuranosyl-4-Imidazolecarboxamide I. Ring Closure with Benzoyl Isothiocyanate,” J. Org. Chem. 32:1825-1828 (1967)) or alternatively, by the method of Clausen (8. Alhede et al., “A Simple and Efficient Synthesis of 9-Substituted Guanines. Cyclodesulfurization of 1-Substituted 5-[(Thiocarbamoyl)amino]imidazole-4-Carboxamides Under Aqueous Basic Conditions,” J. Org. Chem. 56: 2139-2143 (1991)) involving catalysis by a heavy metal salt such as Cu²⁺, Ag⁺, or Hg²⁺ in aqueous NaOH, or, alternatively, by S-oxidation with hydrogen peroxide or sodium perborate in aqueous sodium hydroxide.

Other methods for functionalization of the purine moiety include the use of Michael addition of the 9-nitrogen of the purine moiety to the terminal methylene of an acrylate ester. Another reaction that can be used to functionalize purines is the Mitsunobu reaction. The Mitsunobu reaction is a highly versatile method for the introduction of widely varying functionality upon the purine moiety, because of the wide assortment of primary alcohols that are commercially available for use in this reaction. Mitsunobu chemistry has been shown to be an efficient, specific method for functionalizing purine systems. Mitsunobu conditions were used in the allylation of 2,6-dichloropyrazolopyrimidine and 2,6-dichloropyrrolopyrimidine (A. Dhainaut et al., “New Purines and Purine Analogs as Modulators of Multi-Drug Resistance,” J. Med. Chem. 39: 4099-4108 (1996)). Similarly, 6-chloropyrrolopyrimidine reacted with a primary alcohol (U. Diederichsen & H. W. Schmidt, “β-Homoalanyl-PNA: A Special Case of β-Peptides with β-Sheet-Like Backbone Conformation; Organization in Higher Ordered Structures,” Eur. J. Org. Chem. 1998: 827-835 (1998)). The compound 6-chloropurine also reacted in a similar fashion (M. Iwakawa et al., “Synthetic Routes to Nucleoside Analogs of N-Substituted 1,3-Thiazolidines,” Can. J. Chem. 56:326-335 (1978)). Also, secondary alcohols act as substrates for the Mitsunobu-mediated functionalization of heterocyclic nitrogens.

The application of the Mitsunobu reaction is not limited to halogenated heterocycles such as 6-(N-pyrrolo)purine (K. G. Estep et al., “Synthesis and Structure-Activity Relationships of 6-Heterocyclic-Substituted Purines as Inactivation Modifiers of Cardiac Sodium Channels,” J. Med. Chem. 38:2582-2595 (1995)).

Additionally, a primary heterocyclic amino group does not require protection in the Mitsunobu reaction. The compounds 2-amino-6-benzyloxyguanine (R. E. Dolle & D. McNair, “9-(Sulfoximinoalkyl) Guanine Nucleosides as Potential Antiherpetic Agents,” Tetrahedron Lett. 34:1 (133-136) (1993)) and adenine (S. Van Calenbergh et al., “Synthesis and Structure-Activity Relationship of Analogs of 2′-Deoxy-2′-(3-Methoxybenzamido)adenosine, a Selective Inhibitor of Trypanosomal Glycosomal Glyceraldehyde-3-Phosphate Dehydrogenase,” J. Med. Chem. 38:3838-3849 (1995)) were shown to undergo successful Mitsunobu 9-N-alkylation with no protecting groups needed. However, in some cases, the use of protecting groups such as benzyl or 1-oxy-2-picolyl can be desirable.

Additionally, related methodologies can be used to synthesize N-alkyl substituted analogues such as a selective stepwise bis-amino functionalization procedure (D. A. Nugiel et al., “Facile Preparation of 2,6-Disubstituted Purines Using Solid Phase Chemistry,” J. Org. Chem. 62:201-203 (1997)).

III. Methods of Use of Purine Derivatives And Analogues According to the Present Invention

One aspect of a method of use of purine derivatives and analogues according to the present invention is a method of stimulating regeneration of a mammalian neuron in the peripheral nervous system of a mammal comprising the step of administering an effective amount of a purine derivative or analogue according to the present invention to the mammal.

Another aspect of a method of use of purine derivatives and analogues according to the present invention is a method of stimulating neurogenesis in the central nervous system of a mammal comprising the step of administering an effective amount of a purine derivative or analogue according to the present invention to the mammal.

Yet another aspect of a method of use of purine derivatives and analogues according to the present invention is a method of stimulating neuroprotection in the central or peripheral nervous system of a mammal comprising the step of administering an effective amount of a purine derivative or analogue according to the present invention to the mammal.

Exemplary dosages in accordance with the teachings of the present invention for these purine derivatives and analogues range from 0.0001 mg/kg to 60 mg/kg, though alternative dosages are contemplated as being within the scope of the present invention. Suitable dosages can be chosen by the treating physician by taking into account such factors as the size, weight, age, and sex of the patient, the physiological state of the patient, the severity of the condition for which the purine derivative or analogue is being administered, the response to treatment, the type and quantity of other medications being given to the patient that might interact with the purine derivative or analogue, either potentiating it or inhibiting it, and other pharmacokinetic considerations such as liver and kidney function.

The administration of purine derivatives or analogues according to the present invention is believed to increase the level of mRNA encoding at least one neurotrophic factor that can affect the growth, differentiation, survival, or functioning of neurons in the peripharal or central nervous systems.

The neurotrophic factor can be one of nerve growth factor, NT-3, brain-derived neurotrophic factor (BDNF), or ciliary neurotrophic factor (CNTF); the neurotrophic factor can also be another neurotrophic factor as are well known in the art.

Although Applicants do not intend to be bound by this theory, the increase in the level of mRNA of one or more of these neurotrophic factors brought about by methods according to the present invention employing purine derivatives or analogues according to the present invention is believed to promote neuronal survival.

The term “effective amount” as used herein in this specification means an amount of the purine derivative or analogue that causes a detectable increase in the messenger RNA level of at least one of the recited neurotrophic factors or of another neurotrophic factor known in the art that can be measured. Methods of measuring the mRNA levels typically involve hybridization to probes containing mRNA-specific sequences and detecting the quantity of hybrid nucleic acid formed. The hybrid nucleic acid formed is typically detected by a label incorporated in one of the two nucleic acid strands forming the hybrid. This label can be radioactive or non-radioactive; if non-radioactive, it can be fluorescent, chemiluminescent, bioluminescent, enzymatic, or can make use of another detectable property. Detection can also be performed using the polymerase chain reaction (PCR) mechanism or a variant thereof. PCR is described in detail in U.S. Pat. No. 4,683,195 to Mullis et al. and U.S. Pat. No. 4,683,202 to Mullis et al. Other detection methods, including other amplification methods, are known in the art. One particularly suitable method uses reverse transcription with MMLV reverse transcriptase followed by PCR.

Another method employing purine derivatives and analogues according to the present invention is a method of increasing neuronal function by either inhibiting the formation of the amyloid beta-peptide (Aβ) or stimulating the formation of the secreted derivative of the amyloid precursor protein known as sAPPα by administering to a patient with a neurological disease or a patient at risk of developing a neurological disease an effective quantity of a purine derivative or analogue according to the present invention as described above. The neurological disease can be a neurodegenerative disease, such as, but not limited to, Alzheimer's disease (AD). Alternatively, the neurological disease can be a neurodevelopmental disorder such as, but not limited to, Down's Syndrome.

Yet another aspect of methods according to the present invention is a method of treating peripheral neuropathy comprising administering to a patient with peripheral neuropathy an effective quantity of a purine derivative or analogue according to the present invention. Typically, in this method, the administration of the purine derivative or analogue induces peripheral nerve sprouting in the skin of the patient to whom the purine derivative or analogue is administered. The peripheral nerve sprouting can be nociceptive nerve sprouting. Typically, the nociceptive nerve sprouting is induced without the occurrence of hyperalgesia. The peripheral neuropathy can be diabetic neuropathy or can be a neuropathy associated with the following conditions: acromegaly, hypothyroidism, AIDS, leprosy, Lyme disease, systemic lupus erythematosus, rheumatoid arthritis, Sjögren's Syndrome, periarteritis nodosa, Wegener's granulomatosis, cranial arteritis, sarcoidosis or the administration of other therapeutic agents, e.g. oncolytic drugs

Yet another aspect of the present invention is a method of increasing neuronal function by inducing proliferation of neuronal precursor cells. In general, the method comprises administering to a mammal an effective quantity of a purine derivative or analogue according to the present invention as described above to induce proliferation of neuronal precursor cells in the mammal.

Yet another aspect of the present invention is a method of increasing neuronal function by inducing differentiation of neuronal precursor cells. In general, the method comprises administering to a mammal an effective quantity of a purine derivative or analogue according to the present invention as described above to induce differentiation of neuronal precursor cells in the mammal.

Depending upon the particular needs of the individual subject involved, the purine derivative or analogue according to the present invention may be administered in various doses to provide effective treatment concentrations based upon the teachings of the present invention. What constitutes an effective amount of the selected purine derivative or analogue will vary based upon such factors as the activity of the selected purine derivative or analogue, the physiological characteristics of the subject, the extent or nature of the subject's disease or condition, and the method of administration. Generally, initial doses will be modified to determine the optimum dosage for treatment of the particular mammalian subject. The purine derivative or analogues can be administered using a number of different routes including orally, topically, transdermally, intraperitoneal injection, or intravenous injection directly into the bloodstream. Of course, effective amounts of the purine derivative or analogue can also be administered through injection into the cerebrospinal fluid or infusion directly into the brain, if desired.

The methods of the present invention can be effected using a purine derivative or analogue according to the present invention administered to a mammalian subject either alone or in combination as a pharmaceutical formulation. Further, the purine derivative or analogue according to the present invention can be combined with pharmaceutically acceptable excipients and carrier materials such as inert solid diluents, aqueous solutions, or non-toxic organic solvents. If desired, these pharmaceutical formulations can also contain preservatives and stabilizing agents and the like, as well as minor amounts of auxiliary substances such as wetting or emulsifying agents, as well as pH buffering agents and the like which enhance the effectiveness of the active ingredient. The pharmaceutically acceptable carrier can be chosen from those generally known in the art including, but not limited to, human serum albumin, ion exchangers, dextrose, alumina, lecithin, buffer substances such as phosphate, glycine, sorbic acid, propylene glycol, polyethylene glycol, and salts or electrolytes such as protamine sulfate, sodium chloride, or potassium chloride. Other carriers can be used.

Liquid compositions can also contain liquid phases either in addition to or to the exclusion of water. Examples of such additional liquid phases are glycerin, vegetable oils such as cottonseed oil, organic esters such as ethyl oleate, and water-oil emulsions.

The compositions can be made into aerosol formations (i.e., they can be “nebulized”) to be administered via inhalation. Aerosol formulations can be placed into pressurized acceptable propellants, such as dichloromethane, propane, or nitrogen. Other suitable propellants are known in the art.

Formulations suitable for parenteral administration, such as, for example, by intravenous, intramuscular, intradermal, and subcutaneous routes, include aqueous and non-aqueous isotonic sterile injection solutions. These can contain antioxidants, buffers, preservatives, bacteriostatic agents, and solutes that render the formulation isotonic with the blood of the particular recipient. Alternatively, these formulations can be aqueous or non-aqueous sterile suspensions that can include suspending agents, thickening agents, solublizers, stabilizers, and preservatives. Compositions suitable for use in methods according to the present invention can be administered, for example, by intravenous infusion, orally, topically, intraperitoneally, intravesically, or intrathecally. Formulations of purine derivative or analogues suitable for use in methods according to the present invention can be presented in unit-dose or multi-dose sealed containers, in physical forms such as ampules or vials.

IV. Pharmaceutical Compositions

Another aspect of the present invention is pharmaceutical compositions. A pharmaceutical composition according to the present invention comprises: (1) an effective amount of a purine derivative or analogue according to the present invention as described above; and (2) a pharmaceutically acceptable carrier.

A pharmaceutically acceptable carrier can be chosen from those generally known in the art including, but not limited to, human serum albumin, ion exchangers, alumina, lecithin, buffer substances such as phosphates, glycine, sorbic acid, potassium sorbate, and salts or electrolytes such as potassium sulfate. Other carriers can be used.

The invention is illustrated by the following Examples. These Examples are presented for illustrative purposes only and are not intended to limit the invention.

### EXAMPLE 1

Synthesis of 4-[3-(1-Benzyl-6-oxo-1,6-dihydropurin-9-yl)propionylamino]Benzoic Acid Ethyl Ester

The starting material, 4-[3-(6-oxo-1,6-dihydropurin-9-yl)propionylamino]benzoic acid ethyl ester (400 mg, 0.96 mmol) was suspended in dry N,N-dimethylformamide (10 mL) and then treated with 1,8-diazobicyclo[5.4.0]undec-7-ene (432 [L, 289 mmol). The suspension became a solution with stirring. After 5 minutes, benzyl bromide (228 μL, 1.92 mmol) was added, and the solution stirred at room temperature. A precipitate formed after about 15 minutes. Upon stirring for 24 hours, the reaction mixture was added to ice cold water (50 mL) and the resulting suspension was vacuum filtered washing with water. The isolated solid was dried under vacuum to yield 350 mg (82%) of the title compound.

### EXAMPLE 2

Synthesis of 4-[3-(1-Butyl-6-oxo-1,6-dihydropurin-9-yl)propionylamino]Benzoic Acid Ethyl Ester

The route used in Example 1 was repeated except that butyl iodide replaced the benzyl bromide.

### EXAMPLE 3

Synthesis of 4-[3-(1-Methyl-6-oxo-1,6-dihydropurin-9-yl)propionylamino]Benzoic Acid Ethyl Ester

The route used in Example 1 was repeated except that methyl iodide replaced the benzyl bromide.

### EXAMPLE 4

Synthesis of 4-[3-(1-(2-Dimethylaminoethyl)-6-oxo-1,6-dihydropurin-9-yl)propionylamino]Benzoic Acid Ethyl Ester

The route used in Example 1 was repeated except that 1-chloro-2-dimethylaminoethane replaced the benzyl bromide.

### EXAMPLE 5

Synthesis of 4-[3-(2,6-Dioxo-1,2,3,6-tetrahydropurin-9-yl)propionylamino]Benzoic Acid Ethyl Ester

The starting materials 4-[3-(5-amino-4-carbamoylimidazol-1-yl)propionylamino] benzoic acid ethyl ester (500 mg, 145 mmol), carbonyldiimidazole, (470 mg, 2.9 mmol), and 4-dimethylaminopyridine (18 mg, 0.145 mmol) were combined in dry pyridine (5 mL) and heated to reflux. After 30 minutes a precipitate had formed and the reaction was allowed to cool to room temperature. Then 2 N HCl was added until the pH was about 4-5. The suspension was vacuum filtered washing with dilute HCl. The isolated solid was dried under vacuum to yield 275 mg (51%) of the title compound.

### EXAMPLE 6

Synthesis of 4-[3-(6-Methoxypurin-9-yl)propionylamino]Benzoic Acid Ethyl Ester

In the first step, to a solution of acryloyl chloride (1.08 mL, 13.32 mmol) in dry dichloromethane (10 mL) was slowly added dropwise, via addition funnel, a solution of 4-aminobenzoic acid ethyl ester (2.0 g, 12.11 mmol) and triethylamine (1.86 mL, 13.32 mmol) in dry dichloromethane (5 mL) over about 30 minutes. The reaction was stirred at room temperature for 16 hours. The solution was then partitioned between dichloromethane and water in a separatory funnel. The organic layer was washed with 2 N HCl, dried with sodium sulfate, and concentrated in vacuo to dryness. The resulting residue was triturated with hexane, and vacuum filtered to yield 2.02 g (76%) of 4-acryloylaminobenzoic acid ethyl ester as a solid.

In the second step, 4-acryloylaminobenzoic acid ethyl ester (200 mg, 0.91 mmol), 6-methoxypurine (145 mg, 0.91 mmol), and potassium carbonate (126 mg, 0.91 mmol) were combined in dry N,N-dimethylformamide (3 mL) and stirred at room temperature for 48 hours. Water (20 mL) was added and the resulting precipitate was vacuum filtered washing with water. The isolated solid was dried under vacuum to yield 185 mg (55%) of the title compound.

### EXAMPLE 7

Synthesis of 4-[3-(6-Dimethylaminopurin-9-yl)propionylamino]Benzoic Acid Ethyl Ester

The second step of the procedure of Example 6 was repeated using 6-dimethylaminopurine in place of 6-methoxypurine.

### EXAMPLE 8

Synthesis of 4-[3-(2-Amino-6-chloropurin-9-yl)propionylamino]Benzoic Acid Ethyl Ester

The second step of the procedure of Example 6 was repeated using 2-amino-6-chloropurine in place of 6-methoxypurine.

### EXAMPLE 9

Synthesis of 4-[2-(6-Oxo-2-thioxo-1,2,3,6-tetrahydropurin-9-yl)propionylamino]Benzoic Acid Ethyl Ester

To a suspension of (4-[2-(5-amino-4-carbamoylimidazol-1-yl) propionylamino]benzoic acid ethyl ester (207 mg, 0.599 mmol) in 10 mL pyridine was added phenylisothiocyanate (120 μL, 1.00 mmol). The mixture was stirred and heated at reflux for 5 hours. The mixture was cooled to room temperature and 10 mL of water was added. The resulting precipitate was vacuum filtered, washed with water, and dried under vacuum. To the filtrate was added 75 mL aqueous 2 N hydrochloric acid, bringing the mixture to a pH of about 4. The resulting precipitate was filtered, washed with water, and dried under vacuum. The solid fractions were combined to give 188 mg of the title compound

### EXAMPLE 10

Synthesis of 4-[2-(2-butyl-6-oxo-1,6-dihydropurin-9-yl)propionylamino]Benzoic Acid Ethyl Ester

To a stirred mixture of 4-[2-(5-amino-4-carbamoylimidazol-1-yl)propionylamino]benzoic acid ethyl ester (438 mg, 1.27 mmol) and 2.0 mL N,N-dimethylformamide was added trimethylorthovalerate (400 μL, 2.32 mmol). The solution was heated, and the resultant methanol was distilled off. The reaction was then heated to about 120° C. over 18 hours. The product was purified by flash chromatography (methanol in dichloromethane), yielding 128 mg of the title compound

### EXAMPLE 11

Synthesis of 4-[2-(6-oxo-2-phenyl-1,6-dihydropurin-9-yl)propionylamino]Benzoic Acid Ethyl Ester

The procedure of Example 10 was repeated except that trimethylorthobenzoate replaced the trimethylorthovalerate of Example 10.

### EXAMPLE 12

Synthesis of 4-{[3-(6-Chloropurin-9-yl)propionyl]methylamino}Benzoic Acid Methyl Ester

The first step of Example 6 was repeated using 4-methylaminobenzoic acid methyl ester to obtain 4-(acryloylmethylamino)benzoic acid methyl ester, which was used in the second step with 6-chloropurine to obtain the title compound.

### EXAMPLE 13

Synthesis of 3-(1-Benzyl-6-oxo-1,6-dihydropurin-9-yl)-N-[3-(2-oxopyrrolidin-1-yl)propyl]propionamide

The starting material, 3-(6-oxo-1,6-dihydropurin-9-yl)-N-[3-(2-oxopyrrolidin-1-yl)propyl]propanamide (1000 mg, 3.0 mmol) was suspended in dry N,N-dimethylformamide (15 mL) and then treated with 1,8-diazabicyclo[5.4.0]undec-7-ene (540 μL, 360 mmol). The suspension became a solution with stirring. After 5 minutes, benzyl bromide (430 μL, 3.60 mmol) was added, and the solution stirred at room temperature. A precipitate formed after about 30 minutes. Upon stirring for 24 hours, the reaction mixture was added to acetonitrile (50 mL) and the resulting suspension was vacuum filtered washing with acetonitrile. The isolated solid was dried under vacuum to yield 640 mg (51%) of the title compound.

### EXAMPLE 14

Synthesis of 3-(1-Benzyl-6-oxo-1,6-dihydropurin-9-yl)-N-{2-[2-(2-oxopyrrolidin-1-yl)acetylamino]ethyl}propionamide

The procedure of Example 14 was repeated using 3-(6-oxo-1,6-dihydropurin-9-yl)-N-{2-[2-(2-oxopyrrolidin-1-yl)acetylamino]ethyl}propionamide.

### EXAMPLE 15

Synthesis of N-3-(2-Oxopyrrolidin-1-yl)propyl]-3-(6-oxo-2-thioxo-1,2,3,6-tetrahydropurin-9-yl)propionamide

To a stirred mixture of 5-amino-1-{2-[3-(2-oxopyrrolidin-1-yl)propylcarbamoyl]ethyl}-1H-imidazole-4 carboxylic acid amide (1.01 g, 3.13 mmol) in 20 mL of pyridine was added phenylsothiocyanate (600 1L, 5.02 mmol). The reaction was refluxed for 11 hr and then cooled to room temperature. The pyridine was distilled off under reduced pressure, and the resultant residue was dissolved in 3 mL of methanol and 20 mL of acetonitrile. An additional 10 mL of acetonitrile was added and the resulting precipitate was filtered, washed with methanol, and dried under vacuum to give 602 mg of the title compound.

### EXAMPLE 16

Synthesis of 3-(1-Benzyl-6-oxo-1,6-dihydropurin-9-yl)-N-(3-morpholin-4-yl-propyl)Propionamide

To a suspension of 3-(6-oxo-1,6-dihydropurin-9-yl) propionic acid methyl ester (1.0 g, 4.23 mmol) in 25 mL of dry N,N-dimethylformamide was added 1,8-diazabicyclo[5.4.0]undec-7-ene (1.27 mL, 8.46 mmol) followed by benzyl bromide (0.75 mL, 6.35 mmol) and the reaction stirred at room temperature. After 16 hours, the reaction was partitioned between ethyl acetate and water (100 mL each). The organic layer was separated and washed with saturated aqueous sodium bicarbonate and saturated aqueous sodium chloride (100 mL each), then dried with sodium sulfate and concentrated in vacuo to dryness resulting in 910 mg (66%) of 3-(1-benzyl-6-oxo-1,6-dihydropurin-9-yl) propionic acid methyl ester as a pale yellow liquid. This was used in the next step without further purification.

The 3-(1-benzyl-6-oxo-1,6-dihydropurin-9-yl) propionic acid methyl ester (500 mg, 1.52 mmol) was combined with 3-morpholin-4-yl propylamine (668 mL, 4.57 mmol), and heated to 250° C. with stirring for 3 hours. A precipitate had formed. After cooling, 10 mL of acetonitrile was added and the mixture stirred for 30 minutes. The resulting suspension was filtered, washed with acetonitrile, and dried under vacuum resulting in 200 mg (34%) of the title compound as a white solid.

### EXAMPLE 17

Passive Avoidance Method of Testing Compounds

Passive avoidance is an acute memory paradigm in which mice are allowed to enter a dark compartment from a lighted compartment, but are given a footshock (2 mA for 5 seconds) when they enter the dark compartment. Twenty-four hours after this training session, animals that are placed back in the lighted compartment of two compartment (light-dark) apparatus do not make the transition into the dark compartment. If an amnestic agent (30 mg/kg cycloheximide i.p. in saline) immediately after the training session is given to the animals, they will make the transition into the dark compartment (i.e., memory of the shock is lost). Compounds with suspected nootropic or anti-amnestic effects are given by i.p. administration two hours prior to the training trial in attempt to block the effects of cycloheximide. Mice that exhibit positive nootropic effects are those that avoid moving into the dark chamber. This behavioral response is defined as passive avoidance. A no effect response in this test is defined as a failure to stay in the lighted compartment for 120 seconds.

### EXAMPLE 18

Passive Avoidance Test of 4-[3-(1-Benzyl-6-oxo-1,6-dihydropurin-9-yl)propionylamino]Benzoic Acid Ethyl Ester (Example 1) Administered Intraperitoneally

The passive avoidance test of Example 17 was used to test 4-[3-(1-benzyl-6-oxo-1,6-dihydropurin-9-yl)propionylamino]benzoic acid ethyl ester (Example 1) with intraperitoneal administration with doses ranging from 0.0003 to 10 mg/kg. The minimum effective dose (MED) is 0.0003 mg/kg.

### EXAMPLE 19

Passive Avoidance Test of 4-[3-(1-Benzyl-6-oxo-1,6-dihydropurin-9-yl)propionylamino]Benzoic Acid Ethyl Ester (Example 1) Administered Orally

The passive avoidance test of Example 17 was used to test 4-[3-(1-benzyl-6-oxo-1,6-dihydropurin-9-yl)propionylamino]benzoic acid ethyl ester (Example 1) with oral administration with doses ranging from 0.001 to 1 mg/kg. The MED is 0.01 mg/kg.

### EXAMPLE 20

Passive Avoidance Test of 4-[3-(1-Butyl-6-oxo-1 6-dihydropurin-9-yl)propionylamino]Benzoic Acid Ethyl Ester (Example 2) Administered Intraperitoneally

The passive avoidance test of Example 17 was used to test 4-[3-(1-butyl-6-oxo-1,6-dihydropurin-9-yl)propionylamino]benzoic acid ethyl ester (Example 2) with intraperitoneal administration with doses ranging from 0.001 to 100 mg/kg. The MED is 0.001 mg/kg.

### EXAMPLE 21

Passive Avoidance Test of 4-[3-(1-Methyl-6-oxo-1,6-dihydropurin-9-yl)propionylamino]Benzoic Acid Ethyl Ester (Example 3) Administered Intraperitoneally

The passive avoidance test of Example 17 was used to test 4-[3-(1-methyl-6-oxo-1,6-dihydropurin-9-yl)propionylamino]benzoic acid ethyl ester (Example 3) with intraperitoneal administration with doses ranging from 0.01 to 30 mg/kg. The MED is 0.1 mg/kg.

### EXAMPLE 22

Passive Avoidance Test of 4-[3-(1-(2-Dimethylaminoethyl)-6-oxo-1,6-dihydropurin-9-yl)propionylamino]Benzoic Acid Ethyl Ester (Example 4) Administered Intraperitoneally

The passive avoidance test of Example 17 was used to test the ethyl ester of 4-[3-(1-(2-dimethylaminoethyl)-6-oxo-1,6-dihydropurin-9-yl)propionylamino]benzoic acid ethyl ester (Example 4) with intraperitoneal administration with doses ranging from 0.1 to 100 mg/kg. The MED is 1.0 mg/kg.

### EXAMPLE 23

Passive Avoidance Test of 4-[3-(2,6-Dioxo-1,2,3,6-tetrahydropurin-9-yl)propionylamino]Benzoic Acid Ethyl Ester (Example 5) Administered Intraperitoneally

The passive avoidance test of Example 17 was used to test 4-[3-(2,6-dioxo-1,2,3,6-tetrahydropurin-9-yl)propionylamino]benzoic acid ethyl ester (Example 5) with intraperitoneal administration with doses ranging from 0.00001 mg/kg to 10 mg/kg. The ethyl ester of N-4-carboxyphenyl-3-(2,6-dihydroxypurin-9-yl) propanamide was tested as a suspension in 1% Tween. The MED is 0.001 mg/kg.

### EXAMPLE 24

Passive Avoidance Test of 4-[3-(6-Dimethylaminopurin-9-yl)propionylamino]Benzoic Acid Ethyl Ester (Example 7) Administered Intraperitoneally

The passive avoidance test of Example 17 was used to test 4-[3-(6-dimethylaminopurin-9-yl)propionylamino]benzoic acid ethyl ester (Example 7) with intraperitoneal administration with doses ranging from 0.001 mg/kg to 1 mg/kg. The MED is 0.01 mg/kg.

### EXAMPLE 25

Passive Avoidance Test of 4-[3-(2-Amino-6-chloropurin-9-yl)propionylamino]Benzoic Acid Ethyl Ester (Example 8) Administered Intraperitoneally

The passive avoidance test of Example 17 was used to test 4-[3-(2-amino-6-chloropurin-9-yl)propionylamino]benzoic acid ethyl ester (Example 8) with intraperitoneal administration with doses ranging from 0.00001 mg/kg to 10 mg/kg. The MED is 0.01 mg/kg.

### EXAMPLE 26

Passive Avoidance Test of 4-[2-(6-Oxo-2-thioxo-1,2,3,6-tetrahydropurin-9-yl)propionylamino]Benzoic Acid Ethyl Ester (Example 9) Administered Intraperitoneally

The passive avoidance test of Example 17 was used to test 4-[2-(6-oxo-2-thioxo-1,2,3,6-tetrahydropurin-9-yl)propionylamino]benzoic acid ethyl ester (Example 9) with intraperitoneal administration with doses ranging from 0.00001 mg/kg to 10 mg/kg. The MED is 0.0001 mg/kg.

### EXAMPLE 27

Passive Avoidance Test of 4-[2-(6-Oxo-2-thioxo-1,2,3,6-tetrahydropurin-9-yl)propionylamino]Benzoic Acid Ethyl Ester (Example 9) Administered Orally

The passive avoidance test of Example 17 was used to test 4-[2-(6-oxo-2-thioxo-1,2,3,6-tetrahydropurin-9-yl)propionylamino]benzoic acid ethyl ester (Example 9) with oral administration with doses ranging from 0.001 mg/kg to 1 mg/kg. The MED is 0.001 mg/kg.

### EXAMPLE 28

Passive Avoidance Test of 4-[2-(2-Butyl-6-oxo-1,6-dihydropurin-9-yl)propionylamino]Benzoic Acid Ethyl Ester (Example 10) Administered Intraperitoneally

The passive avoidance test of Example 17 was used to test 4-[2-(2-butyl-6-oxo-1,6-dihydropurin-9-yl)propionylamino]benzoic acid ethyl ester (Example 10) with intraperitoneal administration with doses ranging from 0.01 mg/kg to 10 mg/kg. The MED is 0.1 mg/kg.

### EXAMPLE 29

Passive Avoidance Test of Ethyl Ester of 4-[2-(6-Oxo-2-phenyl-1 6-dihydropurin-9-yl)propionylamino]Benzoic Acid Ethyl Ester (Example 11) Administered Intraperitoneally

The passive avoidance test of Example 17 was used to test 4-[2-(6-oxo-2-phenyl-1,6-dihydropurin-9-yl)propionylamino]benzoic acid ethyl ester (Example 11) with intraperitoneal administration with doses ranging from 0.01 mg/kg to 10 mg/kg. The MED is 10 mg/kg.

### EXAMPLE 30

Passive Avoidance Test of 4-{[3-(6-Chloropurin-9-yl)Propionyl]Methylamino}Benzoic Acid Methyl Ester (Example 12) Administered Intraperitoneally

The passive avoidance test of Example 17 was used to test 4-{[3-(6-chloropurin-9-yl)propionyl]methylamino}benzoic acid methyl ester (Example 12) with intraperitoneal administration with doses ranging from 0.00001 mg/kg to 10 mg/kg. The MED is 0.0001 mg/kg.

### EXAMPLE 31

Passive Avoidance Test of 3-(1-Benzyl-6-oxo-1 .6-dihydropurin-9-yl)-N-[3-(2-oxopyrrolidin-1-yl)propyl]Propionamide (Example 13) Administered Intraperitoneally

The passive avoidance test of Example 17 was used to test 3-(1-benzyl-6-oxo -1,6-dihydropurin-9-yl)-N-[3-(2-oxopyrrolidin-1-yl)propyl]propionamide (Example 13) with intraperitoneal administration with doses ranging from 0.01 mg/kg to 100 mg/kg. The MED is 1.0 mg/kg.

### EXAMPLE 32

Passive Avoidance Test of 3-(1-Benzyl-6-oxo-1,6-dihydropurin-9-yl)-N-{2-[2-(2-oxopyrrolidin-1-yl)acetylaminolethyl}Propionamide (Example 14) Administered Intraperitoneally

The passive avoidance test of Example 17 was used to test 3-(1-benzyl-6-oxo-1,6-dihydropurin-9-yl)-N-{2-[2-(2-oxopyrrolidin-1-yl)acetylamino]ethyl}propionamide (Example 14) with intraperitoneal administration with doses ranging from 0.01 mg/kg to 100 mg/kg. The MED is 1.0 mg/kg.

### ADVANTAGES OF THE PRESENT INVENTION

The present invention provides purine analogues and derivatives that exert a number of biological and physiological functions, particularly increased neuronal function that may involve nerve regeneration in the peripheral nervous system, neurogenesis in the central nervous system, and neuroprotection. The purine analogues and derivatives of the present invention are capable of passing through the blood-brain barrier and exerting their effects in the central nervous system. The components of the purine analogue or derivative can be chosen to optimize the desired activity or range of activities of the molecule and to increase bioavailability.

Although the present invention has been described in considerable detail, with reference to certain preferred versions thereof, other versions and embodiments are possible. Therefore, the scope of the invention is determined by the following claims.

We claim:

1. A purine derivative or analogue having the schematic structure: 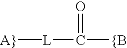
where:
(a) A is a substituted or unsubstituted 9-atom bicyclic moiety in which the five-membered ring has 1 to 3 nitrogen atoms, the bicyclic moiety having the structure of formula (I) 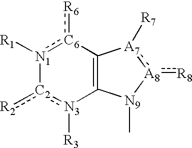
where:
(i) if the bond between N₁ and C₆ is a single bond, then the bond between C₆ and R₆ is a double bond, R₆ is O or S, and R₁ is hydrogen, alkyl, aralkyl, cycloalkyl, or heteroaralkyl;
(ii) if the bond between N₁ and C₆ is a double bond, then the bond between C₆ and R₆ is a single bond, R₁ is not present, and R₆ is hydrogen, halo, amino, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₁ and Q₂ are present together and are alkyl, they can be taken together to form a 5 or 6 member ring which may contain 1 other heteroatom which can be N, O, or S, of which the N may be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S,;
(iii) if the bond between C2 and N₃ is a single bond, then the bond between C₂ and R₂ is a double bond, R₂ is O or S, and R₃ is hydrogen or alkyl;
(iv) if the bond between C₂ and N₃ is a double bond, then the bond between C₂ and R₂ is a single bond, R₃ is not present, and R₂ is hydrogen, alkyl, aralkyl, cycloalkyl, heteroaralkyl, halo, amino, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₁ and Q₂ are present together and are alkyl, they can be taken together to form a 5 or 6 member ring which may contain 1 other heteroatom which can be N, O, or S, of which the N may be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S,;
(v) A₇ and A₈ are C or N;
(A) if A₇ and A₈ are both C and the bond between A₇ and A₈ is a single bond, then the bond between A₈ and R₈ is two single bonds to two hydrogen atoms or is a double bond in which R₈ is O or S and R₇ is two hydrogen atoms;
(B) if A₇ and A₈ are both C and the bond between A₇ and A₈ is a double bond, then R₇ is hydrogen, the bond between A₈ and R₈ is a single bond and R₈ is hydrogen, halo, alkyl, alkenyl, aryl, aralkyl, aralkenyl, heteroaryl, heteroaralkyl, or heteroaralkenyl;
(C) if A₇ and A₈ are both N, then the bond between A₇ and A₈ is a double bond and R₇ and R₈ are not present;
(D) if A₇ is C and A₈ is N, then the bond between A₇ and A₈ is a double bond, R₇ is hydrogen, and R₈ is not present;
(E) if A₇ is N, A₈ is C, and the bond between A₇ and A₈ is a double bond, then R₇ is not present, the bond between A₈ and R₈ is a single bond, and R₈ is hydrogen, halo, alkyl, alkenyl, aryl, aralkyl, aralkenyl, heteroaryl, heteroaralkyl, or heteroaralkenyl;
(F) if A₇ is N, A₈ is C, and the bond between A₇ and A₈ is a single bond, then R₇ is hydrogen, alkyl, aryl, aralkyl, heteroaryl, or heteroaralkyl, the bond between A₈ and R₈ is a double bond, and R₈ is O or S;
(vi) N₉ is bonded to L;
(b) L is a hydrocarbyl moiety of 1 to 6 carbon atoms that can be cyclic, with the hydrocarbyl moiety being optionally substituted with one or more substituents selected from the group consisting of lower alkyl, amino, hydroxy, lower alkoxy, lower alkylamino, lower alkylthio, and oxo; and
(c) B is —OZ or N(Y₁)—D, where Z is hydrogen, alkyl, aryl, heteroaryl, cycloalkyl, aralkyl, or heteroaralkyl, D is a moiety that promotes absorption of the derivative or analogue, and Y₁ is hydrogen, alkyl, aryl, heteroaryl, aralkyl, or heteroaralkyl, which, when taken with D, can form a cyclic 5- or 6-membered saturated structure which can contain one other heteroatom which can be O, N, or S, of which N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;
with the proviso that A does not have the structure of an unsubstituted guanine or hypoxanthine.

2. The purine derivative or analogue of claim 1 wherein A is a purine moiety.

3. The purine derivative or analogue of claim 2 wherein the purine moiety is a purine moiety of Formula (II) 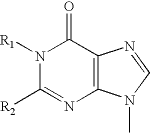
in which:
(a) R₁ is selected from the group consisting of hydrogen, alkyl, aralkyl, cycloalkyl, heteroaralkyl,
(b) R₂ is selected from the group consisting of hydrogen, alkyl, aralkyl, cycloalkyl, heteroaralkyl, halo, amino, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₁ and Q₂ are present together and are alkyl, they can be taken together to form a 5 or 6 member ring which may contain 1 other heteroatom which can be N, O, or S, of which the N may be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S, with the proviso that both R₁ and R₂ are not hydrogen and that R₁ is not hydrogen when R₂ is amino.

4. The purine derivative or analogue of claim 3 wherein R₁ is butyl and R₂ is hydrogen.

5. The purine derivative or analogue of claim 3 wherein R₁ is benzyl and R₂ is hydrogen.

6. The purine derivative or analogue of claim 3 wherein R₁ is dimethylaminoethyl and R₂ is hydrogen.

7. The purine derivative or analogue of claim 3 wherein R₁ is cyclopentyl and R₂ is hydrogen.

8. The purine derivative or analogue of claim 3 wherein R₁ is cyclohexylmethyl and R₂ is hydrogen.

9. The purine derivative or analogue of claim 3 wherein R₁ is cyclopropylmethyl and R₂ is hydrogen.

10. The purine derivative or analogue of claim 3 wherein R₁ is hydrogen and R₂ is phenyl.

11. The purine derivative or analogue of claim 3 wherein R₁ is hydrogen and R₂ is trifluoromethyl.

12. The purine derivative or analogue of claim 3 wherein R₁ is hydrogen and R₂ is butyl.

13. The purine derivative or analogue of claim 3 wherein R₁ is butyl and R₂ is butyl.

14. The purine derivative or analogue of claim 3 wherein R₁ is hydrogen and R₂ is methyl.

15. The purine derivative or analogue of claim 3 wherein R₁ is hydrogen and R₂ is phenylamino.

16. The purine derivative or analogue of claim 2 wherein the purine moiety is a purine moiety of Formula (III), in which: 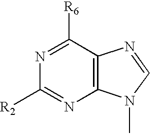
(a) R₂ is selected from the group consisting of hydrogen, halo, amino, OQ₃, SQ₃, NHNH₂, NHOQ₃, NQ₃Q₄, or NHQ₃, where Q₃ and Q₄ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, and heteroaralkylsulfonyl in which the alkyl portions can be cyclic and can contain from 1 to 3 heteroatoms which can be N, O, or S, and when Q₃ and Q₄ are present together and are alkyl, they can be taken together to form a 5- or 6-membered ring which can contain one other heteroatom which can be N, O, or S, of which the N can be further substituted with Y₃, where Y₃ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S; and
R₆ is selected from the group consisting of hydrogen, halo, amino, OQ₅, SQ₅, NHNH₂, NHOQ₅, NQ₅Q₂, or NHQ₆, where Q₅ and Q₆ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, and heteroaralkylsulfonyl in which the alkyl portions can be cyclic and can contain from 1 to 3 heteroatoms which can be N, O, or S, and when Q₅ and Q₆ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S.

17. The purine derivative or analogue of claim 16 wherein R₂ is hydrogen and R₆ is amino.

18. The purine derivative or analogue of claim 17 wherein R₆ is chloro.

19. The purine derivative or analogue of claim 17 wherein R₆ is phenylamino.

20. The purine derivative or analogue of claim 16 wherein R₂ is amino and R₆ is chloro.

21. The purine derivative or analogue of claim 2 wherein the purine moiety is the purine moiety of Formula (IV), below, in which: 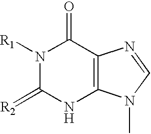
(a) R₁ is hydrogen, alkyl, aralkyl, cycloalkyl, or heteroaralkyl; and
(b) R₂ is O or S.

22. The purine derivative or analogue of claim 21 wherein R₁ is hydrogen.

23. The purine derivative or analogue of claim 22 wherein R₂ is 0.

24. The purine derivative or analogue of claim 22 wherein R₂ is S.

25. The purine derivative or analogue of claim 1 wherein L has the structure —(CH₂)ₙ— wherein n is an integer from 1 to 6.

26. The purine derivative or analogue of claim 25 wherein n is 2.

27. The purine derivative or analogue of claim 25 wherein n is 3.

28. The purine derivative or analogue of claim 1 wherein the moiety B is —OZ.

29. The purine derivative or analogue of claim 28 wherein Z is hydrogen.

30. The purine derivative or analogue of claim 28 wherein Z is alkyl.

31. The purine derivative or analogue of claim 30 wherein Z is selected from the group consisting of methyl, ethyl, butyl, propyl, and isopropyl.

32. The purine derivative or analogue of claim 1 wherein the moiety B is N(Y₁)—D.

33. The purine derivative or analogue of claim 32 wherein Y₁ is hydrogen.

34. The purine derivative or analogue of claim 32 wherein Y₁ is lower alkyl.

35. The purine derivative or analogue of claim 34 wherein Y₁ is methyl.

36. The purine derivative or analogue of claim 32 wherein D is a moiety having at least one polar, charged, or hydrogen-bond-forming group to increase the water-solubility of the purine derivative or analogue.

37. The purine derivative or analogue of claim 36 wherein D is a carboxylic acid or carboxylic acid ester with the structure 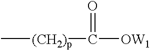
wherein p is an integer from 1 to 6 and W₁ is selected from the group consisting of hydrogen and lower alkyl.

38. The purine derivative or analogue of claim 37 wherein W₁ is hydrogen.

39. The purine derivative or analogue of claim 37 wherein W₁ is ethyl.

40. The purine derivative or analogue of claim 36 wherein D and Y₁ are taken together to form a piperazine derivative of the structure 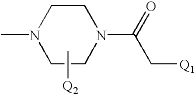
wherein Q₁ is hydrogen, methyl, ethyl, butyl, or propyl, and Q₂ is hydrogen or methyl, where, if Q₂ is methyl, it can be located at either of the two possible positions in the piperazine ring.

41. The purine derivative or analogue of claim 36 wherein D has the structure 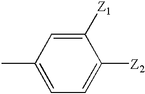
where one of Z₁ and Z₂ is hydrogen, and the other of Z₁ and Z₂ is —COOH or —COOW₁, wherein W₁ is alkyl.

42. The purine derivative or analogue of claim 41 wherein W₁ is selected from the group consisting of methyl, ethyl, propyl, butyl, and isobutyl.

43. The purine derivative or analogue of claim 36 wherein D is a phenylsulfonamidyl moiety of the structure 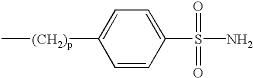
wherein p is an integer from 0 to 6.

44. The purine derivative or analogue of claim 36 wherein D is an alkylpyridyl moiety of structure 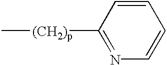
wherein p is an integer from 1 to 6.

45. The purine derivative or analogue of claim 36 wherein D is a dialkylaminoalkyl moiety of the structure 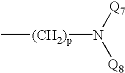
wherein p is an integer from 1 to 6 and Q₇ and Q₈ are alkyl, aralkyl, heteroaralkyl, aryl, heteroaryl, alkanoyl, aroyl, aralkanoyl, heteroaralkanoyl, or heteroaroyl in which the alkyl portions can be cyclic and can contain from 1 to 3 heteroatoms which can be N, O, or S, and when Q₇ and Q₈ are present together and are alkyl, they can be taken together to form a 5 or 6 member ring which may contain 1 other heteroatom which can be N, O, or S, of which the N may be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S.

46. The purine derivative or analogue of claim 45 wherein Q₇ and Q₈ are each alkyl.

47. The purine derivative or analogue of claim 46 wherein Q₇ and Q₈ are each selected from the group consisting of methyl, ethyl, propyl, and isopropyl.

48. The purine derivative or analogue of claim 47 wherein Q₇ and Q₈ are taken together to form a five- or six-membered optionally substituted ring.

49. The purine derivative or analogue of claim 48 wherein the ring is a morpholinyl ring.

50. The purine derivative or analogue of claim 48 wherein the ring is a pyrrolidinyl ring that is optionally substituted with oxo.

51. The purine derivative or analogue of claim 48 wherein the ring is a piperidinyl ring that is optionally substituted with methyl or ethyl.

52. The purine derivative or analogue of claim 36 wherein D is an alkylpyrrolidinyl moiety of the structure 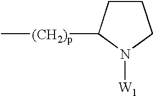
wherein p is an integer from 1 to 6 and W₁ is selected from the group consisting of methyl, ethyl, and propyl.

53. The purine derivative or analogue of claim 1 that has a logP of from about 1 to about 4.

54. A purine derivative or analogue that is 4-[3-(1-benzyl-6-oxo-1,6-dihydropurin-9-yl)propionylamino]benzoic acid ethyl ester.

55. A purine derivative or analogue that is 4-[3-(1-butyl-6-oxo-1,6-dihydropurin-9-yl)propionylamino]benzoic acid ethyl ester.

56. A purine derivative or analogue that is 4-[3-(1-methyl-6-oxo-1,6-dihydropurin-9-yl)propionylamino]benzoic acid ethyl ester.

57. A purine derivative or analogue that is 4-[3-(1-(2-dimethylaminoethyl)-6-oxo-1,6-dihydropurin-9-yl)propionylamino]benzoic acid ethyl ester.

58. A purine derivative or analogue that is 4-[3-(2,6-dioxo-1,2,3,6-tetrahydropurin-9-yl)propionylamino]benzoic acid ethyl ester.

59. A purine derivative or analogue that is 4-[3-(6-methoxypurin-9-yl)propionylamino]benzoic acid ethyl ester.

60. A purine derivative or analogue that is 4-[3-(6-dimethylaminopurin-9-yl)propionylamino]benzoic acid ethyl ester.

61. A purine derivative or analogue that is 4-[3-(2-amino-6-chloropurin-9-yl)propionylamino]benzoic acid ethyl ester.

62. A purine derivative or analogue that is 4-[2-(6-oxo-2-thioxo-1,2,3,6-tetrahydropurin-9-yl)propionylamino]benzoic acid ethyl ester.

63. A purine derivative or analogue that is 4-[2-(2-butyl-6-oxo-1,6-dihydropurin-9-yl)propionylamino]benzoic acid ethyl ester.

64. A purine derivative or analogue that is 4-[2-(6-oxo-2-phenyl-1,6-dihydropurin-9-yl)propionylamino]benzoic acid ethyl ester.

65. A purine derivative or analogue that is 4-{[3-(6-chloropurin-9-yl)propionyl]methylamino}benzoic acid methyl ester.

66. A purine derivative or analogue that is 3-(1-benzyl-6-oxo-1,6-dihydropurin-9-yl)-N-[3-(2-oxopyrrolidin-1-yl)propyl]propionamide.

67. A purine derivative or analogue that is 3-(1-benzyl-6-oxo-1,6-dihydropurin-9-yl)-N-{2-[2-(2-oxopyrrolidin-1-yl)acetylamino]ethyl}propionamide.

68. A purine derivative or analogue that is N-3-(2-oxopyrrolidin-1-yl)propyl]-3-(6-oxo-2-thioxo-1,2,3,6-tetrahydropurin-9-yl)propionamide.

69. A purine derivative or analogue that is 3-(1-benzyl-6-oxo-1,6-dihydropurin-9-yl)-N-(3-morpholin-4-yl-propyl)propionamide.
